(19) United States
(12) Patent Application Publication
(10) Pub. No.: US 2025/0276915 A1
(43) Pub. Date: Sep. 4, 2025

(54) REVERSE OSMOSIS FACILITY

(71) Applicant: B. Braun Avitum AG, Melsungen (DE)

(72) Inventors: Nino Lieb, Melsungen (DE); Philipp Odernheimer, Melsungen (DE)

(21) Appl. No.: 19/065,183

(22) Filed: Feb. 27, 2025

(30) Foreign Application Priority Data
Feb. 29, 2024 (DE) 10 2024 105 880.3

(51) Int. Cl. C02F 1/00; A61M 1/14; A61M 1/16; C02F 1/44; C02F 103/02

(52) U.S. Cl. CPC C02F 1/008; A61M 1/154; A61M 1/1605; C02F 1/441; C02F 2103/026; C02F 2209/008; C02F 2209/02; C02F 2209/03; C02F 2209/05; C02F 2209/40

(57) ABSTRACT

A device for monitoring quality parameters of reverse osmosis process fluids of a reverse osmosis facility includes a control unit with first and second processors. A communication interface transfers data between the first and second processors. The first processor detects and processes a first sensor value, detected by a first sensor, of a first quality parameter of a permeate of the reverse osmosis process fluids. The second processor detects and processes a second sensor value of the first quality parameter of the permeate detected by a further sensor. The control unit calculates a deviation of the first sensor value from the second sensor value and compares the first sensor value and/or the second sensor value with a predeterminable limit value of the first quality parameter. The control unit controls the pump power of at least one pump of the reverse osmosis facility.

### CROSS-REFERENCE TO RELATED APPLICATION

This application claims priority under 35 U.S.C. § 119 to German Application No. 2024 105 880.3, filed on Feb. 29, 2024, the content of which is incorporated by reference herein in its entirety.

### FIELD

The present disclosure relates to a device for monitoring quality parameters of reverse osmosis process fluids of a reverse osmosis facility, a reverse osmosis facility, a method for monitoring quality parameters of reverse osmosis process fluids of a reverse osmosis facility, as well as a computer program and a storage medium comprising the computer program.

### BACKGROUND

Reverse osmosis facilities are known as systems of special filtration or separation technologies and are based on the principle of reverse osmosis

DE202024101009 U1 relates to a reverse osmosis facility. Reverse osmosis is a physical process in which water is pressed through a semi-permeable membrane in order to remove impurities, in particular dissolved solids, ions and molecules. The membrane essentially only allows water to pass through the membrane, while larger particles are retained.

In medical applications, particularly dialysis, the reverse osmosis facility is used for water treatment. In dialysis, a patient's blood is passed outside the body through a machine that takes over the functions of the kidneys by removing waste and excess fluid. As water is an important part of this process, the water must be extremely pure to avoid complications for the patient.

The reverse osmosis facility plays a crucial role in this process by passing the process input water, or any permeate not removed from the ring line, through the membrane(s) to eliminate impurities. The resulting purified water is then used in the dialysis machine to purify the patient's blood.

The use of reverse osmosis ensures that potentially harmful substances such as bacteria, viruses, chemicals and other contaminants are removed from the water. This is crucial to ensure safe and effective dialysis and to minimize the risk of infections or other complications.

It is common in the state of the art to monitor the functionality of the reverse osmosis facility and quality parameters of reverse osmosis process fluids, in particular the quality of the permeate, i.e. after filtration through the membrane. This can be done in particular by monitoring the conductivity of the permeate using a conductivity sensor. The conductivity sensor value is detected by a control unit, which is usually installed inside the reverse osmosis facility. If the control unit registers an increase above a defined limit value via the conductivity sensor, the reverse osmosis facility is stopped. The stop process is realized by stopping the pumps intended for conveying the reverse osmosis process liquids, e.g. a pressure pump and a circulation pump, via the control unit.

The problem here is that in the event of a failure or malfunction of the sensor system or a software error in the control unit, there is no failure compensation and, in particular, no redundancy in the pump shutdown and, if necessary, no possibility of stopping the system. Even if redundancy is created, in particular if a second control unit is provided, the lack of status monitoring and resetting measures cannot be achieved. In devices known in the state of the art, false positive alarms occur during chemical and thermal disinfection, in particular because the conductivities of the permeate can rise above the permitted limit values during these operating phases and a shutdown is therefore unintentional. In addition, longer downtimes, e.g. in standby mode, can also lead to a false positive shutdown, which makes it much more difficult to restart the system.

### SUMMARY

It is therefore an underlying task of the present disclosure to overcome the disadvantages of reverse osmosis facilities in the prior art. In particular, it is an underlying task of the present disclosure to provide reverse osmosis facilities which have improved failure compensation, safety and/or improved condition monitoring.

Preferred embodiments can be taken from the following description.

The embodiments, features and combinations of features as described herein in connection with the present disclosure, but also any combination of features as mentioned and described in connection with the embodiments, are deemed to be disclosed herein, at least as derivable by a person skilled in the art. In particular, each feature and each combination of features in the embodiments described herein may be described, for example, in a different combination, in particular in a different category, at least because the person skilled in the art will recognize that each individual combination of the features mentioned herein is suitable for contributing to the solution of the underlying problem.

Further, each feature and each combination of features in the description below may be used separately. For example, an arbitrary combination selected from one or more embodiments may be provided according to the description below and/or the accompanying figures.

The tasks described above are solved in accordance with the present disclosure by a device for monitoring quality parameters of reverse osmosis process fluids of a reverse osmosis facility. Such a device comprises a control unit with at least a first processor and a second processor. Such a provision of at least two processors of a control unit is particularly advantageous in order to create redundancy and to achieve improved status control. In particular, if one of the processors fails, the second processor can take over (partial) tasks of the failed processor and/or check or detect the status of the other processor. The software of the second processor, possibly the control processor, can be advantageously simple compared to that of the first processor, possibly the main processor. This allows a lower programming effort, memory requirement, hardware structure or similar and may also make sense from a regulatory point of view, e.g. risk assessment, test execution, etc., This is the case because the tasks to be performed by the control system will mostly be of a risk-minimizing nature. It may be advantageous for one processor, preferably the first processor, to assume the function of a main processor and for a second processor to assume the function of a secondary processor. The device according to the present disclosure further comprises a communication interface which is set up for data transfer between the first processor and the second processor. The processors of the device can exchange data via such a communication interface. The two processors preferably work independently of each other, but can also exchange data packets via the communication interfaces. The communication interface is preferably bidirectional, so that data can be transmitted between the processors in both directions. In a preferred embodiment, the processors are essentially similar in their functional and performance scope, but structurally different. The present inventors are convinced that it is best practice to use two processors which are not identical in construction as first and second processors in the present disclosure, since this reduces the risk of a simultaneous failure or a systematic error, possibly caused by the construction series, as far as possible. According to the present disclosure, the first processor, which preferably serves as the main processor, is set up to detect and process a first sensor value, detected by a first quality parameter sensor, of a first quality parameter of a permeate of the reverse osmosis process liquid.

Such a quality parameter is preferably a safety-relevant parameter in the generated permeate, for example the conductivity of the permeate. The second processor is set up to detect and process a further, second sensor value of the first quality parameter of the permeate, which is detected by at least one further quality parameter sensor. The redundant detection of the quality parameter by means of two redundant quality parameter sensors and the independent and redundant transmission and processing of the sensor data by the two processors advantageously improves the operational reliability of the reverse osmosis facility with the device according to the present disclosure. In particular, the independently recorded conductivities of a permeate as quality parameters can advantageously contribute to increasing the safety of the reverse osmosis facility according to the present disclosure, as well as increasing its usability by ensuring the quality of the reverse osmosis process fluids for the intended use. This means that, first of all, the device according to the present disclosure is advantageously suitable for improving the monitoring of the quality of the reverse osmosis process fluids. As a control measure, a status message about the condition of the reverse osmosis facility and/or the reverse osmosis process fluids can be issued to a user. Furthermore, the quality of the reverse osmosis process fluids can then be improved or ensured by initiating corresponding control measures, in particular the control of a physical measure, e.g. a pump or a valve, by the control unit. The decision to take a control measure is based in particular on the processing of the sensor values by the control unit. The device according to the present disclosure is designed in such a way that the control unit is set up to calculate a deviation of the first sensor value from the second sensor value. Additionally or alternatively, the first sensor value and/or the second sensor value is compared with a predeterminable limit value of the first quality parameter.

The term “quality parameter” as used herein preferably refers to a physical parameter of a liquid that is indicative or determinative of the goodness and quality of the liquid. This parameter may characterize various aspects of the fluid relating to its usefulness, purity or other qualitative properties. Examples of quality parameters could include conductivity, temperature, pressure, pH, concentration of certain substances, turbidity, viscosity or other measurable properties of the liquid that are indicative of its composition and suitability for certain purposes. The skilled person will immediately recognize that in the medical application of the device and/or reverse osmosis facility according to the present disclosure, the monitoring and control of quality parameters of the reverse osmosis process fluids is critical to ensure that the fluid meets the required standards and requirements.

The device according to the present disclosure for monitoring the reverse osmosis facility is in particular part of a reverse osmosis facility according to the present disclosure, by means of which the tasks described above can also be solved according to the present disclosure.

The reverse osmosis facility according to the present disclosure is in particular a reverse osmosis facility for medical purposes, which is suitable for being connected to a medical system, in particular at least one medical end device, in particular a dialysis machine, and for supplying it with permeate.

Experts in the field of medical reverse osmosis know that reverse osmosis facilities can be connected in particular to so-called ring mains. Such a ring line known in the prior art is, for example, a supply line within a nephrology center (e.g. a dialysis center, hospital, etc.) to which a large number of dialysis machines or dialysis devices can be connected.

Such a reverse osmosis facility comprises a feed tank which can be supplied with process water via a process input water feed and further comprises a return feed, by means of which return liquid can be fed into the feed tank from a ring line connected to at least one medical terminal device, in particular a dialysis machine. In this context, the term “return liquid” as used herein is preferably understood to mean that permeate that has not been used and has not been removed from the ring line is returned from the ring line. The reverse osmosis facility comprises at least one first filter-pump unit with at least one pump, in particular a pressure pump, which is fluidically connected to the feed tank. The first pump, preferably a pressure pump, is arranged in such a way that the membrane can be supplied with reverse osmosis process fluids from the feed tank by means of the pump to generate the permeate. This generates the permeate as a product and a concentrate as a by-product from the reverse osmosis process fluid in the receiver tank. Furthermore, the reverse osmosis facility comprises a permeate feed line fluidically connected to the at least one membrane, so that the permeate produced can be fed via the ring line to at least one medical terminal device, in particular a dialysis machine. It should be understood that the reverse osmosis facility according to the present disclosure is thus preferably integrated into a circulation system, preferably with such a ring line, and on the one hand supplies permeate for operating the medical terminal devices, in particular dialysis devices, to the circulation system, and in particular to a dialysis device connected to the at least one medical terminal device, a dialysis machine, and on the other hand, unused permeate from the circuit, in particular from a ring line connected to the at least one medical terminal, in particular a dialysis machine, is returned to the storage tank via the return line.

In addition, the reverse osmosis facility can comprise at least one further pump, in particular a circulation pump, which, in order to save water, feeds part of the concentrate back into the process and, in particular, feeds it to the reverse osmosis process liquid from the feed tank located in a feed line to the membrane. Advantageously, the control unit is set up to control the pump power of the first pump and the second pump independently of each other. In particular, the supply of process liquid to the membrane and, alternatively or additionally, the circulation of concentrate can be controlled.

Due to the concentration of substances in the entire circuit of the system, it is particularly necessary to monitor the quality of the process liquid and in particular of the permeate. For this purpose, a first quality parameter sensor for detecting a first sensor value of a first quality parameter of the permeate of the reverse osmosis process liquids is provided in the reverse osmosis facility according to the present disclosure. In addition, at least one further quality parameter sensor is provided for detecting a second sensor value of the first quality parameter of the permeate of the reverse osmosis process fluids. In this way, the first quality parameter, preferably the conductivity, of the permeate can be detected as a quality parameter several times and independently of each other.

The reverse osmosis facility according to the present disclosure preferably also comprises a drain for concentrate and a drain valve connected to the drain. When a control measure is carried out according to the present disclosure, this valve can be opened in order to drain concentrate from the system. In accordance with the present disclosure, the volume of the discharged liquid can be regulated by means of the detection and processing of a first sensor value of a first quality parameter of a permeate of the reverse osmosis process liquids, detected by a first quality parameter sensor, via the quality measure determined in this way. It should be understood that even devices known in the prior art can repeatedly discharge concentrate from the system during normal operation. However, the present disclosure advantageously allows the discarded volume to be adjusted depending on the quality measure detected by the quality parameter sensor. In other words, if the conductivity rises above a limit value, the volume of concentrate discarded via the valve can be increased, if necessary, in order to regulate the conductivity of the process water, in particular to lower it, and thus to create a better condition for permeate with lower conductivity.

According to the present disclosure, the first quality parameter sensor and the at least one further quality parameter sensor are preferably arranged fluidically downstream of the membrane. This means that the first quality parameter of the first reverse osmosis process fluid is detected after passing through the at least one membrane.

The tasks described above are also solved according to the present disclosure by a method according to the present disclosure for monitoring quality parameters of reverse osmosis process fluids. This method is preferably used to monitor safety-relevant parameters in the permeate produced by a reverse osmosis facility. The reverse osmosis facility is preferably a reverse osmosis facility according to the present disclosure or, more preferably, a device according to the present disclosure. The operational reliability of the system is advantageously increased by redundant acquisition and processing of sensor data by two processors. The method according to the present disclosure comprises at least the following steps:

a step a) of detecting a first sensor value of a first quality parameter of a permeate of the reverse osmosis process fluids by means of a first quality parameter sensor and forwarding the first sensor value to a first processor and a step b) of detecting a second sensor value of the first quality parameter of the permeate of the reverse osmosis process fluids by means of a further quality parameter sensor and forwarding the second sensor value to a second processor. It should be understood that the two steps of detecting the first and second sensor values can be carried out independently of each other. Preferably, each detection step is carried out at least once at a predetermined time and/or repeated several times in a predetermined time interval. In this way, the quality of the permeate can be monitored, in particular over longer periods of time and/or at selected points in time, by means of a predetermined, possibly multiple and/or regular detection of the sensor values of the first quality parameter of the permeate. In a step c), data is transferred between the first processor and the second processor by means of the communication interface. In particular, the sensor data is transferred from one processor to the other, either raw or in a form processed by the processors. This exchange can be unidirectional and/or bidirectional between the processors. In particular, a first processor can serve as the main processor and a second processor as a secondary processor. For example, the secondary processor sends the detected/calculated conductivity value to the main processor and/or vice versa. The data transfer takes place via the communication interface.

In a step d) of the method according to the present disclosure, a deviation of the first sensor value from the second sensor value is calculated by means of the first processor and/or the second processor in order to determine, in particular, whether there is a deviation of the first sensor value from the second sensor value which would require the triggering of a control measure. In particular, this makes it possible to determine how close the two measured values are to each other. Since the quality parameter is detected twice independently of each other with the two independent processors, a deviation is particularly indicative of a possible malfunction of one of the sensors and/or one of the processors and/or another function of the reverse osmosis facility.

In order to determine in particular whether one or both of the detected sensor values are in a range that would require the triggering of a control measure, the first sensor value and/or the second sensor value of the first quality parameter of a permeate of the reverse osmosis process liquids is compared with a predetermined maximum sensor limit value in a step f). The predetermined maximum sensor limit value is preferably selected such that a permanent exceeding or falling below of this value in relation to the respective quality parameter is to be avoided. In other words, in addition to comparing the deviation of the quality parameters, for example the conductivities, with each other, the measured and possibly calculated quality parameter, for example the measured/calculated conductivity, is also compared with an absolute value.

The method according to the present disclosure has advantages over the prior art. In particular, the processors can each be checked without the operation of the system having to be interrupted. If, for example, the main processor no longer functions for some reason, for example due to endless loops, too many requests received, too many requests sent, thread frozen, component defects, or the like, this can be recognized by the secondary processor by the fact that the main processor no longer sends and/or requests a sensor value via the communication interface 117. At the same time, the secondary processor 116 can be checked for functionality.

Furthermore, the method according to the present disclosure allows simple detection of sensor defects. Since conductivity is usually measured directly via a current, it is difficult to make a classic distinction between a very low conductivity and a sensor defect, e.g. wire breakage. The independent measuring principle allows a sensor defect to be identified.

Finally, the present disclosure allows the reverse osmosis facility to communicate operating states, for example as a control measure.

The operating phase can be sent via the communication interface and the conductivity monitoring can therefore be switched on and off depending on the operating mode. If an error occurs, the system can also be restarted, for example to remove permeate containing ions from the conductivity probes.

In connection with the method according to the present disclosure, it should also be pointed out that the specified steps do not necessarily have to be carried out in the specified order. The steps indicated can be carried out in any other suitable order or simultaneously.

However, the above sequence may be advantageous for certain embodiments of the method according to the present disclosure. For example, a step a) of detecting a first sensor value can be carried out simultaneously before a step b) of detecting a second sensor value or even simultaneously.

It will also be recognized by one skilled in the art that a feature, embodiment, effect or advantage as described herein in connection with the inventive device and/or the inventive reverse osmosis facility may also be a feature, embodiment, effect or advantage of the inventive process, or vice versa.

In an advantageous embodiment of the device according to the present disclosure, the control unit is set up to switch off the reverse osmosis facility, in particular the at least one first pump or the at least one further pump, depending on the deviation of the first sensor value from the second sensor value, so that the supply of permeate to at least one medical terminal device, in particular a dialysis machine, is prevented. In addition or alternatively, a comparison of the first sensor value and/or the second sensor value with a predeterminable limit value of the first quality parameter can also lead to shutdown as a physical control measure.

In a further advantageous embodiment of the device according to the present disclosure, the control unit is set up to increase the pump power of the at least one first pump and/or the at least one further pump for a predetermined period of time depending on the deviation of the first sensor value from the second sensor value and/or the comparison of the first sensor value and/or the second sensor value with a predeterminable limit value of the first quality parameter of the permeate of the first reverse osmosis process liquid. This advantageously allows the sensor value of the first quality parameter of the permeate measured at the first quality parameter sensor and/or at the at least one further quality parameter sensor to be advantageously reduced.

It should be understood that switching off as a physical control measure represents a safety measure, but prevents the process of supplying the medical end devices, and therefore the patients, with permeate. This can be particularly advantageous if there is a risk of harm to patients based on the behavior of the quality parameters. For example, if an extreme deflection of the quality parameter suggests a permeate quality that would be directly harmful for the introduction of the permeate into the at least one medical terminal device and/or the patient, or if a significantly excessive quality value would be measured over a longer period of time. It will be understood that milder control measures are preferred, which can initially maintain the operation of the at least one medical terminal device. It is particularly preferred to influence the quality of the permeate by means of the device according to the present disclosure and/or the method according to the present disclosure in such a way that it is returned to the predetermined limit values.

In a further advantageous embodiment of the method according to the present disclosure, in a step of taking a control measure based on the result of the comparison in step e) and/or step f), the control unit can take or trigger the control measure. The control measure may comprise the generation of a status message, in particular a status message about the fault-free operation of the reverse osmosis facility or the occurrence of a fault and/or the deviation of a sensor value. Additionally or alternatively, the control unit can also carry out or trigger a physical measure as a control measure.

Such a physical measure can consist of regulating the pump power of a pump, preferably the first pump, in particular a pressure pump, and/or regulating the pump power of another pump, in particular a circulation pump. Additionally or alternatively, a valve, in particular a drain valve for concentrate from the system, can also be controlled or another suitable control measure can be implemented.

In a preferred embodiment of the method according to the present disclosure, the method comprises a step of increasing the pump power of the at least one first pump and/or the at least one pump for a predetermined period of time. It is preferred that the increase is dependent on the deviation of the first sensor value from the second sensor value calculated in step d). Simultaneously or alternatively, the increase is preferably dependent on the comparison in step f) of the first sensor value and/or the second sensor value of the first quality parameter of a permeate of the reverse osmosis process fluids with a predetermined sensor limit value.

In a further advantageous embodiment of the device according to the present disclosure, the device comprises a first printed circuit board (PCB) on which the first processor and the second processor are arranged, preferably by means of galvanic isolation, between the first and the second processor. Additionally or alternatively, processors, in particular the first processor and the second processor, can be arranged on two separate printed circuit boards (PCB).

In a further advantageous embodiment of the device according to the present disclosure, the communication interface is set up for communication with a monitoring unit, wherein the monitoring unit is arranged outside the reverse osmosis facility. This can make it possible to provide a monitoring unit outside the device and, in particular, outside the reverse osmosis facility. For example, the communication interface can communicate with a monitoring unit for wireless communication, in particular via a network connection, Bluetooth or similar. For this purpose, it is advantageous that the communication interface enables data exchange between the monitoring unit and the processors. In particular, the monitoring unit can be part of a computer, a computer program, especially an app.

In an advantageous embodiment of the reverse osmosis facility according to the present disclosure, the reverse osmosis facility comprises at least one further quality parameter sensor for detecting a conductivity and/or a further quality parameter of the reverse osmosis process fluids, in particular the permeate. In turn, the control unit and/or one of the existing or a further processor is set up to record and process the corresponding sensor data. The at least one further quality parameter of the reverse osmosis process fluids is preferably selected from the group comprising conductivity, temperature, volume flow and pressure. In particular, in one embodiment of the present disclosure, the retention can be calculated. The retention is preferably known to the person skilled in the art as the value which indicates how many ions are filtered out of the water. In the context of the present disclosure, a soft water conductivity or process water conductivity (in particular upstream of the membrane) can preferably be measured in order to calculate the retention, wherein a quality sensor for detecting the soft water conductivity is advantageously arranged at the return flow.

The retention can be calculated using the following formula in particular:

$$Formula I$$

$$R \ddot{u} ckhalt [ \% ] = ( 1 - ( LF permeate / LF process input water ) ) * 100 ,$$

where LF Permeate stands for the conductivity of the permeate and LF Process input water for the conductivity of the process input water.

A quality parameter sensor for detecting the conductivity of process input water can be arranged in particular on the supply line of process input water and/or in the supply line area of a storage tank.

The conductivity of the process input water is also preferably measured individually by both processors. Alternatively, the conductivity can also be measured by only one processor and exchanged via the communication interface. This is possible because the conductivity in the process input water is not subject to major fluctuations.

In a further advantageous embodiment of the reverse osmosis facility according to the present disclosure, the at least one further quality parameter sensor for detecting a conductivity and/or a further quality parameter is arranged between the at least one, preferably pressure, pump and/or the at least one further, preferably circulation, pump and the at least one membrane. This advantageously allows the detection of a sensor value of a quality parameter of the reverse osmosis process fluid before contact with the membrane and, if necessary, after an admixture of concentrate.

In a further advantageous embodiment of the reverse osmosis facility according to the present disclosure, the at least one further quality parameter sensor is arranged for detecting a conductivity and/or a further quality parameter for measuring the quality parameter in the process input water.

In a further advantageous embodiment of the reverse osmosis facility according to the present disclosure, at least one further filter-pump unit is provided, which comprises at least one further pump and at least one further membrane, wherein the at least one further filter-pump unit is fluidically connected in series with the at least one first filter-pump unit. In other words, a further pump and a further membrane are arranged downstream of a first membrane and connected downstream of the first membrane.

It should also be understood that as used herein, the term “membrane” preferably refers to a functional unit comprising a single membrane or a plurality of membranes.

In a further advantageous embodiment of the method according to the present disclosure, sensor values of at least one further quality parameter of the reverse osmosis process fluids, in particular of the permeate, are detected by means of a corresponding quality parameter sensor and used in steps c) to f). In other words, in addition to a first quality parameter, for example the conductivity of the permeate, at least one further quality parameter of the reverse osmosis process fluids, for example a temperature of the permeate, or the conductivity of the soft water, is detected or used in the method according to the present disclosure. The at least one further quality parameter of the reverse osmosis process fluids is preferably selected from the group comprising conductivity, temperature, volume flow, pressure.

In particular, the temperature of the reverse osmosis process fluids, especially of the permeate in the process according to the present disclosure, can be detected with a corresponding temperature sensor and used as a quality parameter. In particular, the temperature can be used as an additional quality parameter to the conductivity. The method according to the present disclosure thus advantageously also permits the use of a temperature-compensated conductivity or the temperature can also be additionally monitored as an independent quality parameter according to the method described above.

In a further advantageous embodiment of the method according to the present disclosure, the predetermined deviation limit value is predetermined in step e) as a function of the first sensor value and/or the second sensor value. This allows in particular the realization and use of dynamic limit values in the method according to the present disclosure. In particular in phases with relatively low conductivity, small deviations between the two measurements result in large percentage deviations. To compensate for this, the threshold value of the percentage conductivity can be coupled to the measured conductivity. This can be done either via an equation (e.g. first order) or a look-up table.

The tasks described above are also solved according to the present disclosure by a computer program and/or a storage medium comprising the computer program, wherein the computer program comprises instructions which, when executed by a computer, cause the computer to carry out the method according to the present disclosure.

All described embodiments of the present disclosure have the advantage that in the event of a failure or malfunction of the sensor system or a software error in the control unit, failure compensation and, in particular, redundancy in the pump shutdown is provided. This increases the operational reliability of the reverse osmosis facility. Advantageously, this makes it possible to stop the system even if a sensor and/or a processor were to fail or be impaired. With the device according to the present disclosure, continuous status monitoring can then be achieved and, if necessary, measures can be taken to maintain the operation of the reverse osmosis facility. Advantageously, the occurrence of false positive alarms during chemical and thermal disinfection is also avoided, although the conductivities of the permeate can rise above the permitted limit values during these operating phases and a shutdown is therefore undesirable. Furthermore, the first processor and the second processor can advantageously be easily tested for functionality without having to switch off the reverse osmosis facility according to the present disclosure.

It is therefore an underlying task of the present disclosure to overcome the disadvantages of reverse osmosis facilities in the prior art. In particular, it is an underlying task of the present disclosure to provide reverse osmosis facilities which have improved failure compensation, safety and/or improved condition monitoring.

### BRIEF DESCRIPTION OF THE DRAWING FIGURES

The present disclosure is explained in more detail below with reference to the drawings, from which further features, embodiments and advantages can be seen. In the embodiments shown in the figures, elements which have similar or identical functions are provided with the same reference signs. It should be noted that the illustrations may not be to scale.

FIG. 1 shows a device according to the state of the art with a sensor system and a processor;

FIG. 2 shows another prior art device with two independent sensors and independent processors;

FIGS. 3A and 3B show embodiments of the present disclosure;

FIG. 4 shows a schematic sequence of the process steps of the method according to the present disclosure in a first embodiment; and

FIG. 5 shows a measurement of the conductivity as a quality parameter in a further embodiment of the method according to the present disclosure.

### DETAILED DESCRIPTION

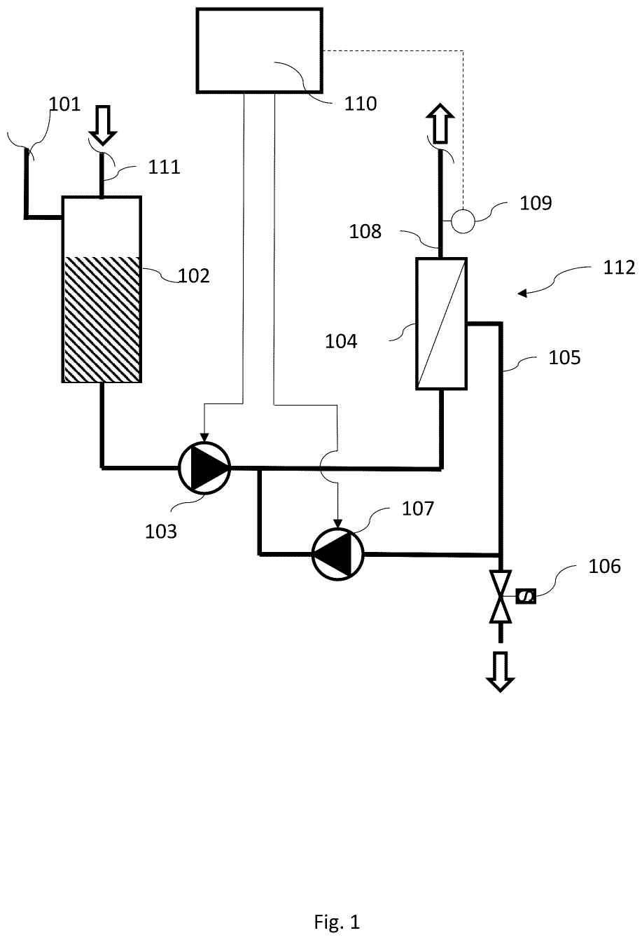

FIG. 1 shows a schematic representation of a reverse osmosis facility according to the prior art. These systems are generally constructed in such a way that the process input water 101 is fed into a storage tank 102. Then a pressure pump 103 draws the required water from the feed tank 102 and presses it into the membranes 104. This process produces permeate 108 as a product and concentrate 105 as a by-product. To save water, some of the concentrate 105 can be fed back into the process via a circulation pump 107. In order to transport some of the retained ions out of the system, the solenoid valve 106 is opened to transport concentrate 105 into the wastewater.

The permeate 108 is fed into a ring line where it can be taken from one or more medical end devices, for example dialysis machines. Unused permeate 108 is returned to the feed tank 102 via the return line 111. As a rule, the conductivity of the permeate 108 is detected as a quality parameter of the permeate 108 of the osmosis process fluid by a first quality parameter sensor 109, usually a conductivity sensor 109, in order to monitor the filtering process. The measurement and possibly processing of the sensor values are carried out by a control unit 110, which is within the reverse osmosis facility 112. If the control unit 110 registers an increase above a defined limit value via the conductivity sensor 109, the reverse osmosis facility 112 is stopped. The stopping process is realized by stopping the pressure pump 103 and the circulation pump 107 via the control unit 110. It is obvious that in the event of a malfunction of the sensor 109 or the control unit 110, stopping the pumps is the only intended control measure. This means that a simple malfunction, a software error or similar leads to an immediate stop of the pumps, even if the actual conductivity of the permeate 108 has not increased. On the other hand, if the shutdown did not occur and the control unit 110 had a corresponding software error or the conductivity sensor 109 had a defect, a problem in the pump shutdown or other could also occur and there would be no way to stop the system.

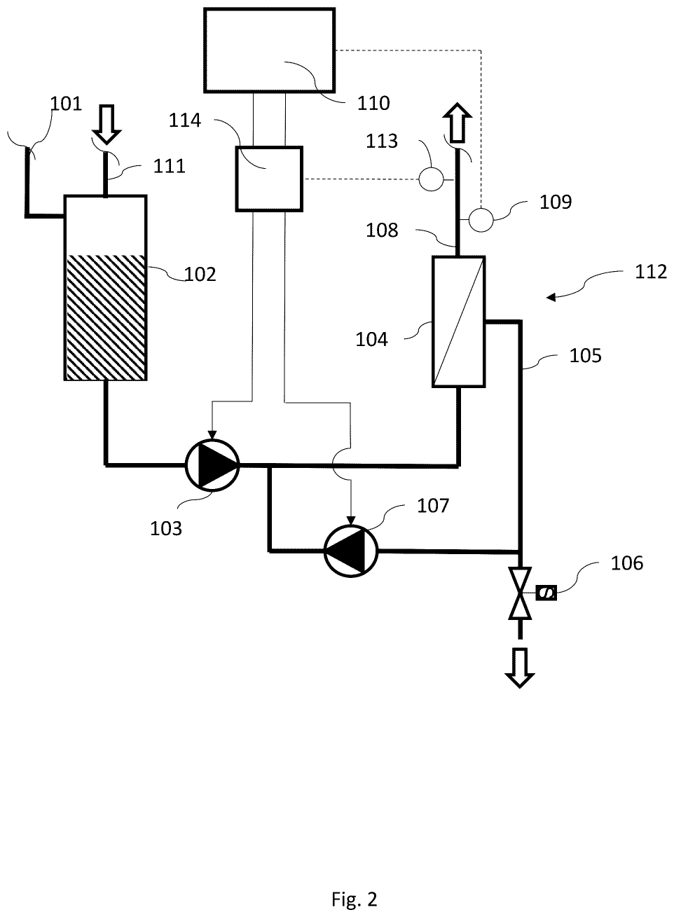

FIG. 2 shows a schematic illustration of a further development of the reverse osmosis facility shown in FIG. 1 that is known in the prior art. Here, a further measuring device independent of the primary control unit 110 is installed in the reverse osmosis facility 112, which also monitors the quality of the permeate 108 with a conductivity sensor 113. The secondary control unit 114 has a second processor, which is set up to detect and process a second sensor value of the conductivity detected by at least one further quality parameter sensor 113 as the first quality parameter of the permeate 108. If this secondary control unit 114 detects an increase in conductivity above a permitted limit value, the reverse osmosis facility 112 is stopped as described above in connection with FIG. 1. Here, the objective of the secondary control unit 114 is to compensate for a potential failure of the primary control unit 110 and thus to achieve failure compensation compared to the system shown in FIG. 1 with only one sensor system and only one control unit.

However, a disadvantage of the prior art with second control unit 114 shown in FIG. 2 is the lack of status control and the lack of possibility for resetting measures. In other words, the second control unit 114 has no information about the current operating mode and, in particular, no information about the function of the first control unit 110 and the first sensor 109. This can lead to false positive alarms, in particular during chemical and thermal disinfection. The conductivities can rise above the permitted limit values during these operating phases and thus lead to an unintentional shutdown. A false positive error can also occur due to a longer service life. Ions begin to diffuse into the permeate 108 due to the unpressurized state at the membrane. This leads to an increase in conductivity in the permeate 108 depending on the osmotic pressure and time. As soon as the limit value of the secondary control unit 114 has been exceeded, the systems can no longer be started. As a result, the fault cannot be rectified by the operator and a technically trained person must restart the system with major intervention. During these times, it is not possible to operate the medical end devices, and in particular no treatments in the dialysis center. In addition, the implementation of the prior art shown in FIG. 2 is usually cost intensive in practice. In the prior art, the software is usually switched to an off mode when an error occurs and the user is notified of the problem. The user may then have the option of performing a rinse mode, preferably for a limited time (˜1 min), or alternatively restarting a dialysis mode to verify the error. If the membrane is intact, the permeate can be flushed away with high conductivity by these measures and the system can be operated as usual. If the membrane is defective, the conductivity remains high and the fault remains active. Usually, in the state of the art, the conductivity is not monitored in this mode, as no patients are treated with the device during these operating phases. Therefore, data exchange is also relevant (exchange of operating states). In general, the conductivity rises sharply during these operating phases.

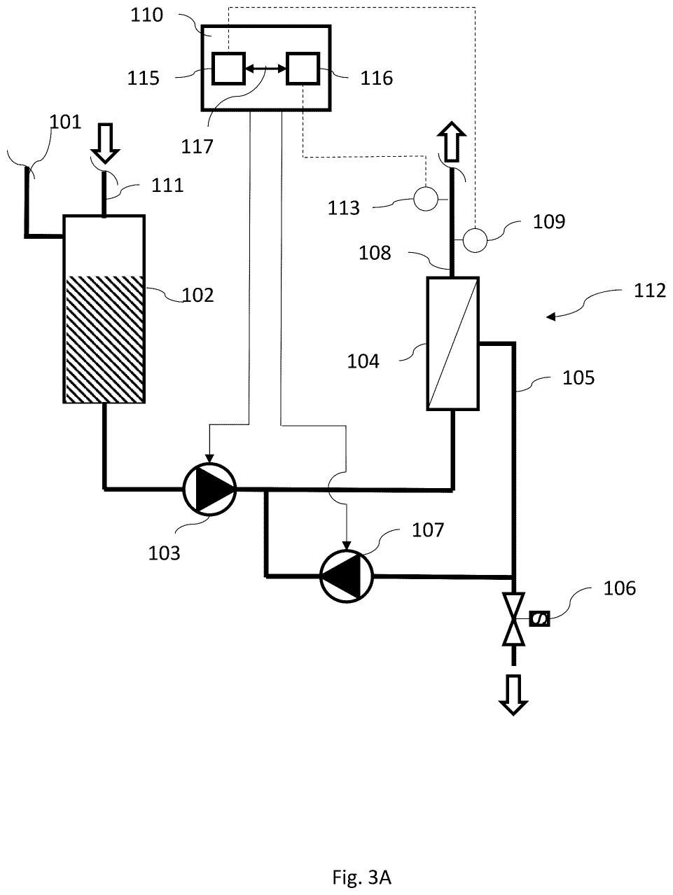

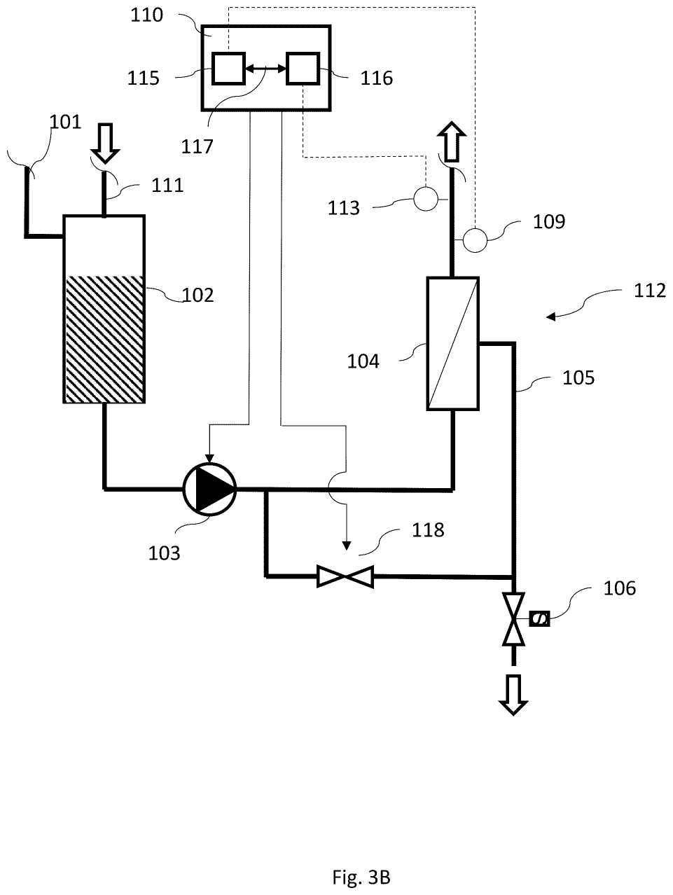

FIG. 3 shows a reverse osmosis facility 112 according to an embodiment of the present disclosure. A feed tank 102 is provided, which can be supplied with process water via a process input water feed 101 and further comprises a return feed 111, by means of which return liquid from a ring line connected to the at least one medical terminal device, in particular dialysis device, can be fed into the feed tank 102 (arrow pointing downwards on the left in the figure). Also provided is a filter-pump unit, with a pressure pump 103 and a circulation pump 107, as well as a membrane unit with one or more membranes 104, which are fluidically connected to the supply tank 102 and serve to generate the permeate 108 from the reverse osmosis process fluids of the supply tank 102. The control unit 110 is arranged to control the pump power of the first pump 103 and the second pump 107 independently of each other. As in the prior art in FIG. 1 and FIG. 2, the first pump 103 is arranged such that the membrane 104 can be supplied with reverse osmosis process fluid from the supply tank 102 by means of the pump 103 to generate the permeate 108, and the permeate 108 can then be supplied to the at least one medical terminal device, in particular dialysis machine, by means of the permeate supply line (arrow pointing upwards on the right in the figure) via the ring line. A first quality parameter sensor 109 for detecting a first sensor value of a first quality parameter of the permeate 108 of the reverse osmosis process fluids, preferably the conductivity of the permeate, and at least one further quality parameter sensor 113 for detecting a second sensor value of the first quality parameter of the permeate 108 of the reverse osmosis process fluids detect the quality parameter. The reverse osmosis facility in FIG. 3A and FIG. 3B, respectively, further comprises a device for monitoring the reverse osmosis facility 112 according to the present disclosure. Thus, in contrast to the prior art, a second control unit 114 is absent. In this regard, the device according to the present disclosure includes a control unit 110 comprising at least a first processor 115 and a second processor 116, as well as a communication interface 117 configured to transfer data between the first processor 115 and the second processor 116. The first processor 115 detects and processes a first sensor value of a first quality parameter of a permeate 108 of the reverse osmosis process fluids, for example the conductivity, detected by a first quality parameter sensor 109. At the same time, the second processor 116 independently records and processes a second sensor value of the first quality parameter of the permeate 108 detected by at least one further quality parameter sensor 113. The control unit 110 calculates a deviation of the first sensor value from the second sensor value and compares both sensor values independently of one another with a predeterminable limit value of the first quality parameter. Thus, on the one hand, it is determined whether the sensor values essentially match or deviate from one another. On the other hand, it is determined whether the sensor values are within the predeterminable limit values. Limit values can be set sensibly depending on the selected parameter. For example, in the case of conductivity, it makes sense to set an upper limit value, whereas it may make sense to set upper and lower limits for temperature. It should also be understood that the method according to the present disclosure or the device according to the present disclosure for monitoring are particularly advantageous in a state during the treatment of a patient. For example, monitoring would advantageously be suspended during a disinfection operation. This advantageously restricts monitoring to times at when a patient is exposed to a potential hazard. Likewise, at times when a patient is not exposed to a potential hazard, e.g. during a cleaning or disinfection phase, sensor values can be permitted that would otherwise lead to an alarm or stop of the system.

The systems shown in FIGS. 3A and 3B differ in the presence of the second pump 107 in the embodiment shown in FIG. 3A. It should be made clear from FIG. 3B that the device according to the present disclosure can also be used advantageously in systems without such a second pump 107. In particular, it is possible to feed the concentrate directly to a drain valve 106. The skilled person knows that recirculation is mainly carried out to save water and to avoid so-called “dead-end” filtration, which reduces the longevity of the membranes. As an alternative to the pump 107, a valve 118, in particular in the form of a needle valve, can also be used at the corresponding point, which can optionally also be controlled by the control device 110. Usually, instead of or in addition to controlling the valve 118, manual control is provided, for example by means of a corresponding manual valve.

Switching off the pressure pump 103 is sufficient to stop the reverse osmosis process, as there are no other energy generators in the system.

The control unit 110 thus initiates a control measure if either the deviation is outside a tolerance range or the sensor values deviate too much from the predeterminable limit value of the first quality parameter. Such a control measure can comprise the generation of a status message and/or the control of a physical measure, for example a control of the pump power of a pump, preferably the pump 103 and/or the pump 107, or the control of a valve, preferably a drain valve 106. This allows in particular the operation of the reverse osmosis facility according to the present disclosure with an average low pump power. Although a high pump power, and thus possibly an increased liquid volume flow, generally leads to an improved permeate quality, a low pump power is desirable, in particular in order to save operating energy and thus enable more environmentally friendly and cost-effective operation. However, at a low pump speed, as well as with a long service life, an undesirable accumulation can occur in the permeate, which increases the conductivity. In accordance with the present disclosure, a control measure can be triggered here as a control measure and the pump power can be increased for a limited time, whereby the enrichment can be reduced and the conductivity of the permeate can be quickly normalized. Similarly, smaller measurement outliers can be compensated for. It is possible to create an escalating control measure scenario and, for example, to follow up a first measure, e.g. the temporary increase in pump power, with a second control measure, especially if the measured values do not normalize. It is also possible to specify a time window, e.g. 30 s, for the effect of a first control measure. The hierarchy of processors, namely a first main processor and a second secondary processor, can also be used particularly advantageously here, whereby, for example, the main processor takes an escalating control measure with a delay, e.g. after 60 s, and in extreme cases shuts down the system. The conductivity of the permeate 108 is also the preferred quality parameter in the device and reverse osmosis facility according to the present disclosure, as the conductivity is a good indicator of the quality of the permeate, i.e. in particular the concentration of impurities, pollutants, bacteria, viruses and other undesirable substances.

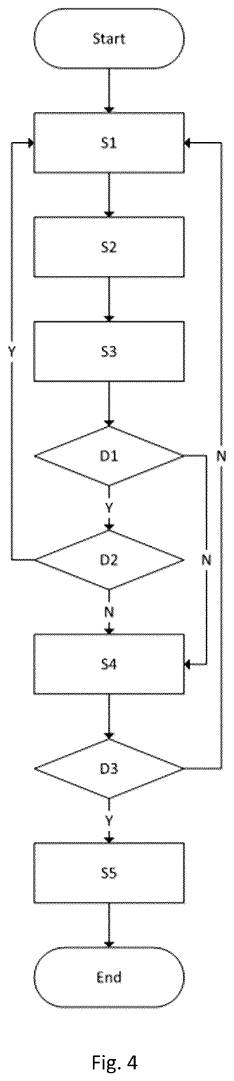

FIG. 4 shows a schematic flow chart for the method according to the present disclosure. The method is suitable for monitoring quality parameters of reverse osmosis process fluids of a reverse osmosis facility, as shown for example in FIG. 3A or FIG. 3B. In simple terms, in the method according to the present disclosure, the two independently recorded conductivities are compared with each other and must not exceed a defined deviation. Furthermore, both main processor 115 and secondary processor 116 compare the conductivity against an absolute limit value.

Shown as S1 is a step a) of detecting a first sensor value of a first quality parameter of a permeate 108 of the reverse osmosis process fluids by means of a first quality parameter sensor 109 and forwarding the first sensor value to a first processor 115 and a step b) of detecting a second sensor value of the first quality parameter of the permeate 108 of the reverse osmosis process fluids by means of a further quality parameter sensor 113 and forwarding the second sensor value to a second processor 116.

In other words, the main processor 115 records the transmitted measured value of the conductivity sensor 109 and the secondary processor 116 records the transmitted measured value of the conductivity sensor 113. This sequence may also include the calculation of the conductivity if the sensor does not have an integrated transmitter.

Shown as S2 is a step c) of the data transfer between the first processor 115 and the second processor 116 by means of a communication interface 117. In other words, the main processor 115 sends the detected/calculated conductivity value to the secondary processor 116 and vice versa. The data transfer takes place via the communication interface 117.

In the illustrated step S3, the deviation of the first sensor value from the second is calculated in step d). In principle, this calculation can be carried out by both processors. However, the main processor 115 can also perform the calculation alone. The deviation is preferably calculated according to the following formula.

$$Deviation [ \% ] = [ ( C 1 - C 2 ) / C 1 ] * 100 \qquad Formula II$$

where C1=conductivity measured at a first quality parameter sensor 109, and where C2=conductivity measured at a further quality parameter sensor 113 The calculation can also be carried out similarly by the secondary processor 116 and is shown below.

$$Deviation [ \% ] = [ ( C 1 - C 2 ) / C 2 ] * 100 \qquad Formula III$$

In a step e) referred to as D1, the deviation determined in step d) is compared with a predetermined deviation limit value. In this way, it is determined whether the deviation is within an allowed range. Again, this calculation can be performed by only one of the two processors and/or the main processor 115 and the secondary processor 116 compare the respectively calculated deviation against a permissible deviation independently of each other. The value of the permissible deviation is preferably predetermined identically for both processors.

In step f), shown as D2, the first sensor value and preferably the second sensor value of the first quality parameter of a permeate 108 of the reverse osmosis process fluids is compared with a predetermined sensor limit value. In other words, it is determined whether the conductivities are within the permitted range. In addition to comparing the deviation of the conductivities with each other in step D1, or e), the measured/calculated conductivity is also compared against an absolute value. Preferably, the main processor 115 compares the value measured at the conductivity probe 109 against the limit value. Additionally or alternatively, the secondary processor 116 compares the value measured at the conductivity probe 113 against the limit value. Here again, the predetermined sensor limit value is preferably identical for both processors. An advantageous application is to compare a low measured conductivity against a fixed limit value, for example less than or equal to 50 μS/cm, possibly with a corresponding tolerance of about +/−5 μS/cm. Additionally or alternatively, larger conductivities, for example greater than 50 μS/cm, can also be compared with a tolerance of approximately 10% deviation.

The present method according to the present disclosure can thus be used to advantageously monitor the function and the quality of the reverse osmosis process fluids, in particular the conductivity of the permeate 108.

In addition, the present disclosure can advantageously carry out control measures itself. In particular, in a step designated as step S4 in the diagram, such a control action can be performed, in which the control action is performed by the control unit 110 based on the result of the comparison in step e) or D1 and/or step f) or D2, and wherein the control action comprises generating a status message and/or controlling a physical action.

In particular, measures can be taken to reduce conductivity.

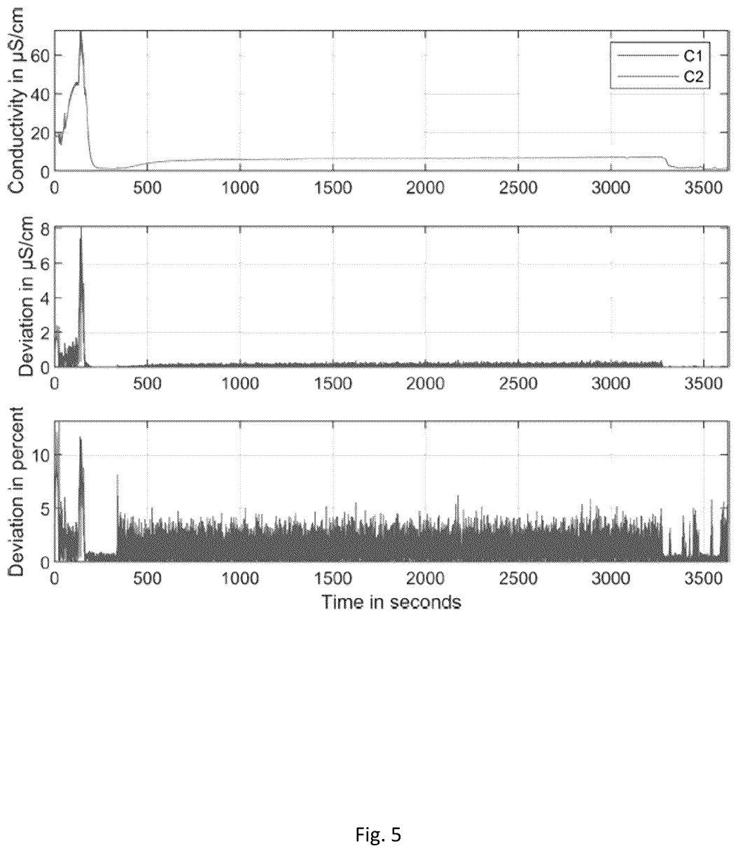

Such a measure is particularly effective and fast by means of the present disclosure, as shown in FIG. 5. There, the conductivity C1 measured at a first quality parameter sensor 109, and the conductivity C2, measured at a further quality parameter sensor 113, are each shown in μS/cm (upper graph), as well as the deviation calculated according to formula II in μS/cm (middle graph) and in [%], each over the time in seconds. As can be seen immediately, the conductivity rises steeply at around 100 seconds and is addressed by the method according to the present disclosure with a control measure after around 200 seconds from the start of the measurement. Firstly, the speed of the pressure pump 103 is increased. This ensures that fewer ions can diffuse through the membrane, as the working pressure increases compared to the osmotic pressure. In addition, permeate 108, into which more ions have diffused, is flushed out of the membrane modules 104, which leads to a rapid normalization of the conductivity. Potential ion accumulations in the electrical field of the conductivity probes 109 and 113 can also be removed as a result. In addition, the yield was reduced in this case and the solenoid valve 106 was opened for a limited time so that more concentrate 105 is discharged from the system. As a result, ion-containing concentrate 106 is replaced by process input water 101. The conductivity of the permeate 108 is therefore also reduced while the filter performance remains the same. As can be seen directly from the graphs in FIG. 5, these physical control measures take effect within just a few seconds and the conductivity of the permeate 108 measured with quality parameter sensor 109 and quality parameter sensor 113 drops to normal values. It should also be noted that during proper operation and full functionality of the quality parameter sensors 109 and 113, the corresponding measured values C1 and C2 are almost identical, as there are hardly any differences in the curve.

As described above, a de-escalating control measure scenario is implemented in step D3 by providing a delay time in which the aforementioned control measures, increasing the pump power and opening the valve 106, can take effect before the system is switched off, i.e. the pressure pump 103 and the circulation pump 107 in particular are switched off in step S5, unless the measured values normalize within a predetermined time window, the delay time. This also allows short-term trends to be buffered. It should be noted that the delay time for the error of the deviation and the exceeded limit value can be the same, but in a preferred embodiment are not the same.

Furthermore, it should be noted that the trigger time for main processor 115 and secondary processor 116 may be the same, but in a preferred embodiment are unequal, as described further above. The main processor 115 should preferably have a lower trigger time than the secondary processor 116. Furthermore, the control device can be set up so that the secondary processor can only react if the main processor fails. It is advantageous that a differentiation between the two types of faults and the two processors is permitted. This is an advantage when identifying and rectifying faults.

The embodiments shown in the figures may relate to preferred embodiments, while all elements and features described in connection with the embodiments, as appropriate, may be used in combination with any other embodiment and feature as discussed herein, particularly with respect to any other embodiment discussed above.

The features of the present disclosure disclosed in the description, examples and/or figures may be material for realizing the present disclosure in various forms, both individually and in any combination thereof.

### Reference Character List

101-Process input water
102-Feed tank
103-Pressure pump
104-Diaphragm(s)
105-Concentrate
106-Solenoid valve
107-Circulation pump
108-Permeate
109-Conductivity sensor
110-Control unit
111-Return
112-Reverse osmosis facility
113-Conductivity sensor
114-second control unit
115-Main processor
116-Auxiliary processor
117-Communication interface
118-Needle valve

1. A device for monitoring quality parameters of reverse osmosis process fluids of a reverse osmosis facility, the device comprising:
a control unit with at least a first processor and a second processor;
a communication interface, set up for data transfer between the first processor and the second processor;
the first processor being set up to detect and process a first sensor value of a first quality parameter of a permeate of the reverse osmosis process fluids, detected by a first quality parameter sensor; and
the second processor being set up to detect and process a second sensor value of the first quality parameter of the permeate, detected by at least one further quality parameter sensor,
the control unit being arranged for calculating a deviation of the first sensor value from the second sensor value and for comparing the first sensor value and/or the second sensor value with a predeterminable limit value of the first quality parameter, and
the control unit being set up to initiate at least one control measure of the reverse osmosis facility to control pump power of at least one first pump or at least one second pump of the reverse osmosis facility.

2. The device according to claim 1, wherein the first quality parameter is a conductivity of the permeate.

3. The device according to claim 1, wherein the control unit is set up, depending on the deviation of the first sensor value from the second sensor value and/or a comparison of the first sensor value and/or the second sensor value with a predeterminable limit value of the first quality parameter, to switch off the reverse osmosis facility so that a supply of permeate to at least one medical terminal device is prevented.

4. The device according to claim 1, wherein the control unit is set up to increase pump power of the at least one first pump and/or the at least one second pump for a predetermined period of time, depending on the deviation of the first sensor value from the second sensor value and/or a comparison of the first sensor value and/or the second sensor value with a predeterminable limit value of the first quality parameter of the permeate of the reverse osmosis process fluids.

5. The device according to claim 1, further comprising a first printed circuit board, wherein the first processor and the second processor are arranged on the first printed circuit board.

6. The device according to claim 1, further comprising a first printed circuit board and a second printed circuit board, wherein the first processor is arranged on the first printed circuit board and the second processor is arranged on the second printed circuit board.

7. The device according to claim 1, wherein the communication interface is set up for communication with a monitoring unit, wherein the monitoring unit is arranged outside the reverse osmosis facility.

8. A reverse osmosis facility comprising:
the device according to claim 1;
a feed tank configured to be supplied with process water via a process water supply inlet, the feed tank comprising a return feed by which return liquid is feedable into the feed tank from a ring line connected to at least one medical terminal; and
at least one first filter-pump unit having a first pump, a second pump and a membrane, the at least one first filter-pump unit being fluidically connected to the feed tank for generating a permeate from the reverse osmosis process fluids of the feed tank,
the control unit of the device configured to control the pump power of the first pump and the second pump independently of one another,
the first pump being arranged such that the membrane is configured to be supplied with reverse osmosis process fluids from the feed tank by the first pump to produce the permeate,
a permeate feed line being fluidically connected to the membrane, so that the permeate produced is feedable via the ring line to at least one medical terminal device, and
a first quality parameter sensor being configured to detect a first sensor value of a first quality parameter of the permeate of the reverse osmosis process fluids and at least one further quality parameter sensor for detecting a second sensor value of the first quality parameter of the permeate of the reverse osmosis process fluids, the first quality parameter preferably being a conductivity of the permeate.

9. The reverse osmosis facility according to claim 8, wherein the at least one first filter-pump unit further comprises at least one membrane.

10. The reverse osmosis facility according to claim 8, wherein the at least one medical terminal device is a dialysis machine.

11. The reverse osmosis facility according to claim 8, further comprising at least one further quality parameter sensor for detecting a conductivity and/or a further quality parameter of reverse osmosis process liquids, wherein the further quality parameter is selected from the group consisting of conductivity, temperature, volume flow, and pressure.

12. The reverse osmosis facility according to claim 8, wherein the at least one further quality parameter sensor for detecting a conductivity and/or a further quality parameter is arranged between the first pump and the membrane, or is arrange between the second pump and the membrane and/or the at least one further quality parameter sensor for detecting a conductivity and/or a further quality parameter is arranged for measuring the quality parameter in process input water.

13. The reverse osmosis facility according to claim 8, further comprising at least one further filter-pump unit comprising at least one second pump and at least one further membrane, wherein the at least one further filter-pump unit is fluidically connected in series with the at least one first filter-pump unit.

14. A method for monitoring quality parameters of reverse osmosis process fluids of the reverse osmosis facility according to claim 8,
the method comprising at least the steps of:
a) detecting a first sensor value of a first quality parameter of a permeate of the reverse osmosis process fluids by means of a first quality parameter sensor and forwarding the first sensor value to a first processor,
b) detecting a second sensor value of the first quality parameter of the permeate of the reverse osmosis process fluids by means of a further quality parameter sensor and forwarding the second sensor value to a second processor,
c) transferring data between the first processor and the second processor by means of a communication interface;
d) calculating a deviation of the first sensor value from the second;
e) comparing the deviation determined in step d) with a predetermined deviation limit value; and
f) comparing the first sensor value and/or the second sensor value of the first quality parameter of a permeate of the reverse osmosis process fluids with a predetermined sensor limit value.

15. The method according to claim 14, wherein the first quality parameter is a conductivity of the permeate.

16. The method according to claim 14, further comprising the step of taking a control action in which, based on a result of a comparison in step e) and/or step f), the control action is taken by the control unit.

17. The method according to claim 14, further comprising at least one of the following steps:
increasing the pump power of the first pump or the second pump for a predetermined period of time; or
in step f), comparing the first sensor value and/or the second sensor value of the first quality parameter of a permeate of the reverse osmosis process fluids with a predetermined sensor limit value.

18. The method according to claim 14, wherein sensor values of at least one further quality parameter of the reverse osmosis process fluids are detected by corresponding quality parameter sensors and used in steps c) to f), wherein the at least one further quality parameter of the reverse osmosis process fluids is selected from the group consisting of conductivity, temperature, volume flow, and pressure.

19. The method according to claim 14, wherein the predetermined deviation limit value is predetermined in step e) depending on the first sensor value and/or the second sensor value.

20. A computer program and/or a storage medium comprising the computer program, wherein the computer program comprises instructions which, when executed by a computer, cause the computer to perform the method according to claim 14.
